(19) United States
(12) Patent Application Publication
(10) Pub. No.: US 2024/0353405 A1
(43) Pub. Date: Oct. 24, 2024

(54) Automated Bacterial Cell Counting Devices, Systems and Methods Thereof

(71) Applicants: Agency for Science, Technology and Research, Singapore (SG); Singapore Heavy Engineering Pte Ltd, Singapore (SG)

(72) Inventors: Laura SUTARLIE, Singapore (SG); Xiaodi SU, Singapore (SG); Alan Jenting SHE, Singapore (SG); Khin Moh Moh AUNG, Singapore (SG)

(21) Appl. No.: 18/682,794

(22) PCT Filed: Aug. 8, 2022
(86) PCT No.: PCT/SG2022/050567

§ 371 Date: Feb. 9, 2024

(30) Foreign Application Priority Data
Aug. 12, 2021 (SG) 10202108807U

(51) Int. Cl. G01N 33/569; G01N 33/543; G01N 35/10

(52) U.S. Cl. CPC G01N 33/56911; G01N 33/54386; G01N 35/1002; G01N 35/1067; G01N 35/1095

(57) ABSTRACT

The disclosure concerns bacterial cell counting devices, systems and methods thereof. The bacterial cell counting device comprises at least one cartridge for containing reagents; an inlet for introducing a sample containing bacterial cells into the device; an optofluidic chip separately in fluid communication with the cartridge and the inlet; a filter strip passing through the optofluidic chip and in fluid communication with the cartridge and the inlet, the filter strip for trapping or retaining bacterial cells on its surface such that the bacterial cells can interact with the reagents as they flow through the filter strip; and a controller for controlling a sequential flow of reagents and sample to the filter strip via the optofluidic chip. The optofluidic chip is capable of detecting a colorimetric and/or fluorescence output emitted from the bacterial cells modified by the reagents in order for the bacterial cells to be quantified relative to a control.

### CROSS-REFERENCE TO RELATED APPLICATIONS

This patent application is a US National Phase Entry under 371 National Stage Entry of 35 U.S.C. § 371 of co-pending International Patent Application No. PCT/SG2022/050567 filed Aug. 8, 2022, titled “Automated Bacterial Cell Counting Devices, Systems and Methods Thereof”, which claims priority to Singaporean patent application Ser. No. 10/202,108807U filed Aug. 12, 2021, the contents of which are incorporated by reference herein, for all purposes, in its entirety.

### TECHNICAL FIELD

The present invention relates, in general terms, to automated bacterial cell counting devices, systems and the methods thereof.

### BACKGROUND

Bacterial contamination is of general concern in many areas, including environment, food, (bio) pharmaceutical processes, agriculture/aquaculture, and medical processes. The current “standard” method for bacteria detection is by plate culture. It is time consuming (2-7 days) and cannot detect unculturable and/or inactive bacteria. The plate culture also highly relies on lab facilities and trained personnel.

Enzymatic methods takes about 18-44 h but highly relies on lab facilities and trained personnel. The bacteria also needs to be pre-cultured. There is also no specificity to bacteria. The main disadvantage of enzyme-based methods is that the bacteria detection relies on enzymatic activity of the enzymes in bacteria cells. Enzyme reaction is known to be highly affected by pH and temperature. This enzymatic ATP reaction is also temperature sensitive, and not specific to bacteria (the source of ATP can be from non-bacteria substances).

Molecular methods by detecting bacteria genetic materials like polymerase chain reaction takes several steps of bacterial lysis, gene extraction followed by enzymatic polymerase chain reactions. This method is quite tedious, and requires highly trained personnel, expensive equipment and laboratory facilities.

Current bacteria testing methods are either culture-based methods that take a long time, or faster molecular/enzymatic methods that is quite tedious and require trained personnel.

Developing rapid microbiological methods (RMM) has attracted high interest. Currently there is no automated RMM that are culture-free and enzyme-free.

It would be desirable to overcome or ameliorate at least one of the above-described problems.

### SUMMARY

The present invention relates to bacteria counting devices, systems and methods thereof, for the continuous and sequential detection of bacteria concentration in water.

The device can be an automated device. This device consists of integrated machine hardware and electronics, which can be used together with data acquisition software and process protocols (or programmable recipes) to form a system to detect and count bacterial cells. Specifically, the device and system allow for continuous bacteria counting, through automated sequential processes of drawing water samples, filtering the bacteria cells on a membrane, drawing the nanoparticle reagents to stain the bacterial cells on the membrane, measuring the colour intensity, and reporting the quantity of the bacterial cells in the water samples. To achieve this, various scientific and engineering challenges have been overcome in order to enable the aspects of automation, repeatability, compactness, and user-friendliness.

The present invention provides a bacterial cell counting device, comprising:

a) at least one cartridge for containing reagents;

b) an inlet for introducing a sample containing bacterial cells into the device;

c) an optofluidic chip separately in fluid communication with the cartridge and the inlet;

d) a filter strip passing through the optofluidic chip and in fluid communication with the cartridge and the inlet, the filter strip for trapping or retaining bacterial cells on its surface such that the bacterial cells can interact with the reagents as they flow through the filter strip; and

e) a controller for controlling a sequential flow of reagents and sample to the filter strip via the optofluidic chip;

wherein the optofluidic chip is capable of detecting a colorimetric and/or fluorescence output emitted from the bacterial cells modified by the reagents in order for the bacterial cells to be quantified relative to a control.

As will be understood, a controller will generally be embodied by electronic components, particularly electronic components programmed to facilitate control of a sequential flow of reagents and sample into a bacterial cell counting device, and other necessary functions.

In some embodiments, the bacterial cell counting device is automated.

In some embodiments, the cartridge is replaceable.

In some embodiments, the cartridge comprises an aqueous nanoparticle solution.

In some embodiments, the aqueous nanoparticle solution comprises a plasmonic and/or fluorescent nanoparticle and a surfactant, the nanoparticle functionalised with positively charged molecules and/or affinity probes for binding to said bacterial cells trapped or retained on the surface of the filter strip via charge recognition and/or affinity binding.

In some embodiments, the bacterial cell counting device comprising a second cartridge, wherein the cartridge comprises an aqueous nanoparticle solution comprising a plasmonic and/or fluorescent nanoparticle functionalised with positively charged molecules and a surfactant; and wherein the second cartridge comprises an aqueous nanoparticle solution comprising a plasmonic and/or fluorescent nanoparticle functionalised with affinity probes and a second surfactant.

In some embodiments, the bacterial cell counting device further comprises a washing tank, the washing tank comprising an aqueous solution for washing the filter strip.

In some embodiments, the optofluidic chip is a microfluidic chip.

In some embodiments, the optofluidic chip further comprises at least one of the following:

i) clamping and unclamping means for securing and releasing the filter strip;

ii) means for chamber pressurization;

iii) means for channel mixing; and

iv) purging means.

In some embodiments, the filter strip is provided as a filter tape in a filter cassette.

In some embodiments, the filter tape is re-usable by winding to an adjacent unused region about 3 cm apart from a used region.

In some embodiments, when the filter tape comprises at least two used region, an earlier used region is recallable for measurement by winding the filter tape to that earlier used region.

In some embodiments, the filter strip is a PVDF membrane having a pore size of about 0.22 μm.

In some embodiments, the bacterial cell counting device further comprises a waste collection tank for containing the reagents and sample after elution from the optofluidic chip.

In some embodiments, the bacterial cell counting device further comprises an optical detector for measuring the colorimetric and/or fluorescence output emitted from the bacterial cells modified by the reagents.

In some embodiments, the control is a calibration plot.

In some embodiments, the bacterial cell counting device is characterised by a limit of detection of about 1000 CFU/mL.

The present invention also provides a bacterial cell counting system, comprising:

a) the bacterial cell counting device as disclosed herein; and

b) a control system operable to automatically flow the reagents and sample sequentially to the filter strip via the optofluidic chip.

The present invention also provides a method counting bacterial cells in a sample using a bacterial cell counting device as disclosed herein, comprising:

a) flowing the sample in a liquid form through the filter strip, the filter strip for trapping or retaining bacterial cells on its surface thereof;

b) flowing a first aqueous nanoparticle solution through the filter strip, the first aqueous nanoparticle solution comprising a plasmonic and/or fluorescent nanoparticle and a first surfactant, the nanoparticle functionalised with positively charged molecules and/or affinity probe for binding to said bacterial cells trapped or retained on the surface of the substrate via charge recognition and/or affinity binding; and

c) flowing a second aqueous solution through the filter strip, the second aqueous solution for washing the unbound nanoparticle from the porous substrate;

wherein the bacterial cells in the sample is quantifiable by a colorimetric and/or fluorescence output emitted from the nanoparticle bound to the bacterial cells trapped or retained on the filter strip.

In some embodiments, the method further comprises a step before step a) of purging the filter strip with a wetting solution for removing residual reagents in at least the optofluidic chip.

In some embodiments, the method further comprises a step after step a) of washing filter strip in order to remove non-bacterial cell particulates.

In some embodiments, the method further comprises a step after step b) of incubating the first aqueous nanoparticle solution with the bacterial cells trapped or retained on the filter strip.

In some embodiments, the bacterial cells in a sample can be quantified within about 1 min to about 30 min.

### BRIEF DESCRIPTION OF THE DRAWINGS

Embodiments of the present invention will now be described, by way of non-limiting example, with reference to the drawings in which:

FIG. 1. (A) Schematic of the Bacterial Cell Counting Device. Five components are labelled, namely (1) nanoreagent cartridge, (2) automated sampler, (3) optofluidic chip, (4) filter cassette with filter tapes, (5) control and measurement electronics. (B) a photograph of the device. (C) a photograph of a filter tape cassette.

FIG. 2. Test of the PVDF membrane suitability before cutting and loading to the cassette. The membrane (white color) is cut and assembled on the cap of an injection vial (a) before passing through gold nanoparticles (AuNPs) reagent solution, (b) after passing through gold nanoparticles reagent and thus stained, (c) after washing with water, remains white without red colour from AuNPs. The membrane is hydrophilic (can pass through AuNPs and water) and does not bind nanoparticles.

FIG. 3. Testing of the detector on marker pen stained PVDF filter membrane

FIG. 4. (Left) the stained PVDF membrane in the cassette with varied E. coliconcentration. (Right) The transmission intensity of the respective stains.

FIG. 5. Full Automation test results. (Left) the stained PVDF membrane in the cassette with varied E. coli concentration. (Right) The transmission intensity of the E. coli samples of different concentrations.

FIG. 6. The transmission intensity for different E. coli concentration from the fully automated 26 samples.

FIG. 7. The transmission intensity for the continuous 50 automated samples. Each data point represents one sample measurement.

FIG. 8. Real water test results by (A) the bacterial cell counting device and (B) Plate culture.

### DETAILED DESCRIPTION

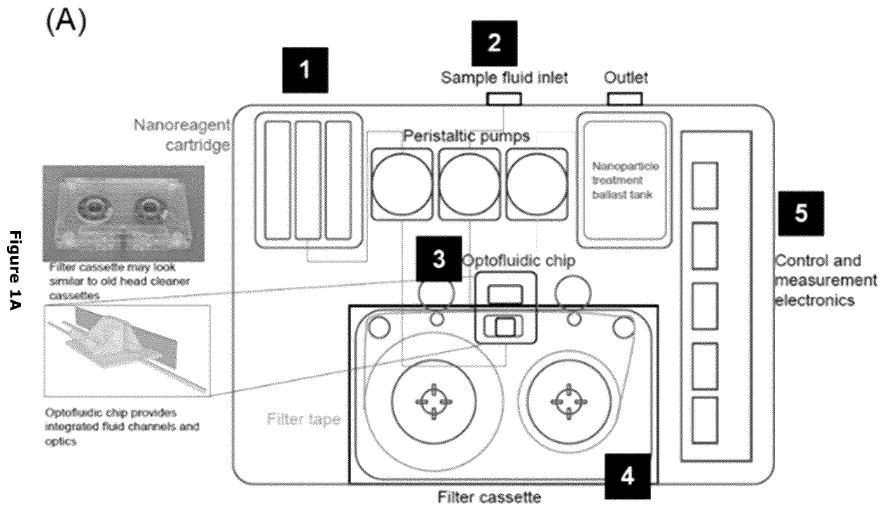

The bacterial cell counting device comprises five components: (1) nanoreagent cartridge, (2) inlet which may comprise an automated sampler, (3) optofluidic chip, (4) filter cassette with filter tapes, (5) control and measurement electronics. The control electronics or controller is configured to sequentially flow the reagents and sample. These components can be hosted in a compact compartment such as a chip (FIG. 1). The bacterial cell counting device may be operatable by a control system. With properly designed process protocols, the system can perform sample filtering, staining, and measurement of bacteria load in water samples by using nanoparticles reagent, without hand-on actions.

As used herein, “device” refers to a thing or entity made or adapted for a particular purpose, such as a piece of mechanical or electronic equipment. The device can be manually operated, or can computer implemented with instructions from a software.

As used herein, “system” refers to one or more devices configured with or interacting with each other based on a set of rules. The set of rules can be provided by a software and/or process protocols. In this sense, a system is a group of interacting or interrelated elements that act according to a set of rules to form a unified whole. A system, surrounded and influenced by its environment, is described by its boundaries, structure and purpose and expressed in its functioning.

As used herein, “method” refers to a particular procedure for accomplishing or approaching something. Accordingly, and with reference to this invention, when a particular set of rules is selected, the system can provide a method for automatically detecting and quantifying bacterial cells.

Accordingly, the present invention provides a bacterial cell counting device, comprising:

a) at least one cartridge for containing reagents;

b) an inlet for introducing a sample containing bacterial cells into the device;

c) an optofluidic chip separately in fluid communication with the cartridge and the inlet;

d) a filter strip passing through the optofluidic chip and in fluid communication with the cartridge and the inlet, the filter strip for trapping or retaining bacterial cells on its surface such that the bacterial cells can interact with the reagents as they flow through the filter strip; and

e) a controller for controlling a sequential flow of reagents and sample to the filter strip via the optofluidic chip;

wherein the optofluidic chip is capable of detecting a colorimetric and/or fluorescence output emitted from the bacterial cells modified by the reagents in order for the bacterial cells to be quantified relative to a control.

The present device can be controlled manually where fine control of reagents and reaction time is required. The present device also provides the advantage of an automated, culture-free, and enzyme-free bacteria counting system, direct bacteria sample measurement without sample treatment, continuous and programmable operation, and with a Limit of Detection (LOD) of 1000 CFU/mL. This automated bacteria-counting system is highly suitable for real-time measurement on-site of various environmental conditions where plate culture is impossible and enzyme based rapid tests are impractical due the inconsistent enzyme activity due to environment condition changes. For example, this system can be used to monitor bacteria contamination in areas where human operation is expensive and impractical, for example in applications like remote and programmed site monitoring and inspection.

In addition, other advantages include:

1. By changing the nanoreagent content in the cartridge, the system can detect an unlimited range of different bacteria species

2. By installing multiple nanoreagent cartridges of different contents, the system can detect multiple bacteria species in a programmable manner

3. The filter strip can be contained within a cassette, the design of which allows automated filter feeding (by moving the filter spot connected to the optofluidic chip) without end user to change the filter every measurement, and automated storing of the filter after staining.

The present invention can be applied in bacteria contamination detection, remote detection of water quality, on-line assessment of water treatment outcome, machine health monitoring (good bacteria count) for biological treatment systems, such as membrane bioreactor (MBR) water treatment units, and aquaculture water assessment for bacteria count related to disinfection treatment.

As mentioned, the bacterial cell counting device comprises an inlet. The inlet is for introducing a sample containing bacterial cells into the device. In some embodiments, the inlet comprises an auto sampler. An auto sampler is a device that provides samples for analysis. The sample size can be pre-determined and fixed to a certain volume. In this way, consistency across all the tested samples can be achieved and the amount of bacterial cells can be quantified. The auto sampler can comprise multiple mini-sized piezo electric pumps with digital control.

The inlet (and the auto sampler) can be configured to provide a fixed and consistent amount of sample to the bacterial cell counting device. To further improve consistency, a larger amount of sample can be aliquoted, from which a subset can be drawn for detection and quantification.

The volume of sample may be determined by an interplay between factors such as minimizing time needed to analyse one sample and the largest amount of reagent that can be pumping through the filter strip within a reasonable amount of time. In some embodiments, for industrial water and environmental water, a volume of less than about 1 ml is aliquoted. In some embodiments, for drinking water, a volume of more than about 5 mL is aliquoted. Drinking water is cleaner than environmental water and hence 5 mL or more may be preferred so as to increase the detection sensitivity, and without the filter strip being clogged.

The inlet can further comprise a filter. The filter can be a 5 μm pore-size filter, for removing high solid particulate background from environmental samples.

The inlet can further comprise mixing means. The mixing means ensures that the sample is homogenous before it is aliquoted. This provides for a consistent and accurate sampling as bacterial cells are denser than water and can sediment over time.

The bacterial counting device comprises at least one cartridge. The cartridge is for containing reagents. The reagents react with the bacterial cells to provide a detectable response. In some embodiments, at least one component in the reagents bind to the bacterial cells. The reagents can be a nanoparticle solution (or nanoreagent). In some embodiments, the cartridge comprises an aqueous nanoparticle solution. For example, component can be nanoparticle. The nanoparticle can be a plasmonic and/or fluorescent nanoparticle, such as metal nanoparticles. Metal nanoparticles include (but is not limited to) Au nanoparticle, Ag nanoparticle, Au core-Pt shell nanoparticles, and semiconductor quantum dots. Carbon based luminescent nanoparticles can also be used. For example, Au nanoparticle (AuNP) can be functionalised to bind to the bacterial cells. The Au nanoparticles can be surface functionalised with ligands, linkers, proteins, amino acids, carbohydrates, antibodies, or a combination thereof such that they preferably bind to bacterial cells instead of the filter strip. Alternatively, the nanoparticles can have an affinity for bacterial cells as a result of the functionalisation. In this way, a more accurate quantification can be obtained when unbound nanoparticles are removed by a rinsing step.

In some embodiments, the nanoparticle is functionalised with positively charged molecules and/or affinity probes for binding to said bacterial cells trapped or retained on the surface of the filter strip via charge recognition and/or affinity binding.

In some embodiments, the aqueous nanoparticle solution comprises a plasmonic and/or fluorescent nanoparticle and a surfactant. In some embodiments, the aqueous nanoparticle solution comprises a plasmonic and/or fluorescent nanoparticle and an ionic surfactant. The ionic surfactant acts to stabilise the nanoparticles and prevent aggregation and/or agglomeration. This can result in a false positive as the large aggregates cannot be rinsed away easily. The ionic surfactant may be a cationic surfactant for forming charge-based nanoparticles, such as cetyltrimethylammonium bromide (CTAB) and cetyltrimethylammonium chloride (CTAC). Nonionic surfactant for forming affinity-based nanoparticles such as Tween-20 can also be used.

In addition to nanoparticles as mentioned above, the component (or reagent) can for example be an affinity probe tagged with a fluorescent protein. The affinity probe can be a biomolecule such as protein, antibody, or aptamer which may target bacterial cells in general or a specific bacteria species.

The bacterial counting device can be configured with multiple cartridges, or at least 2 cartridges. The cartridges can contain different contents for multiplexing in a programmable manner. For example, a second cartridge can contain a further different aqueous nanoparticle solution. In this regard, when the first cartridge comprises a first aqueous nanoparticle solution comprising a plasmonic and/or fluorescent nanoparticle functionalised with positively charged molecules and a first surfactant, the second cartridge can comprise a second aqueous nanoparticle solution comprising a plasmonic and/or fluorescent nanoparticle functionalised with affinity probes and a second surfactant. In other embodiments, the first and second aqueous nanoparticle solutions each have nanoparticles with different plasmonic and/or fluorescent emissions, and the nanoparticles are functionalised with different affinity probes. This allows the detection and quantifications of mixtures of bacterial cells in the sample.

The surfactant in the second cartridge can be a different surfactant (second surfactant) from the surfactant in the first cartridge. In some embodiments, the surfactant in the second cartridge can be a different ionic surfactant (second ionic surfactant) from the ionic surfactant in the first cartridge. Using different surfactants depending on the reagents improves the stability of the reagents. Alternatively, the surfactants in both the first and second cartridge can be the same. This can be advantageous in preventing or minimising osmotic shock to the bacterial cells.

In some embodiments, the cartridge is replaceable. Alternatively, the cartridge can be refilled with the reagent when necessary.

The bacterial cell counting device comprises an optofluidic chip. The optofluidic chip is fluidly communicated with the cartridge(s), and separately fluidly communicated with the inlet. In some embodiments, the optofluidic chip is a microfluidic chip. The optofluidic chip is configured to allow the sample and reagents from the cartridge to flow through its fluidic channel, and also to allow for colorimetric and/or fluorescence detection.

With regard to flowing of fluids through the optofluidic chip, the bacterial cell counting device also comprises a filter strip passing through the optofluidic chip. The filter strip is in fluid communication with the cartridge and the inlet. The filter strip is positioned such that it traps or retains bacterial cells on its surface when the sample from the inlet flows through the optofluidic chip and the filter strip. In this way, as the reagent also flows through the filter strip, bacterial cells can interact with the reagents.

In some embodiments, the optofluidic chip further comprises clamping and unclamping means for securing and releasing the filter strip. This prevents the filter strip from being displaced during the analysis. The clamping and unclamping means can engage the filter strip a substantial and/or pre-determined distance away from the sampling area, or for example about 3 to 10 cm away from the sampling area. This prevent contamination.

The filter strip together with the optofluidic chip forms a tight seal such that fluids from the sample and the reagents do not leak. Rubber clamps clamping from both sides of the filter strip may be used. The rubber clamps may be configured such that they fit with the channel diameter.

In some embodiments, the optofluidic chip further comprises means for chamber pressurization. Additionally, chamber pressurization may be used to push the fluid through the filter. In some embodiments, a pressure may be used to facilitate a flow of large or sticky substances in the fluid. The pressure may be increased by using a fluid pump.

In some embodiments, the optofluidic chip further comprises means for channel mixing. The means may be configured to mix bacteria suspended in the sample, and/or mix the reagent.

In some embodiments, the optofluidic chip further comprises purging means. The purging means releases any air bubbles within the microfluidic channels to allow for accuracy in the sampling. The purging means may further be configured to release an aqueous medium from a washing tank into the fluidic channel to clean the optofluidic chip.

In some embodiments, the filter strip is replaceable. Following a sampling, the filter strip is stained with reagents and bacterial cells. This filter strip can be replaced with a new filter strip and a new sample can be tested for bacterial cells. To improve it usability, in some embodiments, the filter strip is provided as a filter tape in a filter cassette. In this regard, the filter tape is windable to an adjacent unused region at a pre-determined distance or about 3 cm apart from a used region. A clean region of the filter tape is hence exposed to the optofluidic chip and sampling can proceed.

For example, after the first spot on the filter tape is used for staining, the filter tape position can be shifted to expose a new area. The distance between the first spot and the next one is optimized so that there is no overlapping staining from previous sample, but not too far to better utilize the filter tape. The optimized distance may be 3 cm.

By providing the filter strip in the form of a filter tape, information regarding the sample is stored and can be recalled if necessary. Measurements can be retested through this recollection. In some embodiments, when the filter tape comprises at least two used region, an earlier used region can be recalled by winding the filter tape to that earlier used region for measurement.

In some embodiments, the filter strip is a PVDF membrane having a pore size of about 0.22 μm.

In some embodiments, a standard audio cassette is used to host the PVDF filter membrane to make a “filter tape cassette” to provide the function of introducing a new filter for each measurement.

For example, the filter cassette can be:

(a) of a standardized size and be interchangeable with a new cassette;

(b) loaded with 0.22 μm PVDF membrane, which has been selected and validated for its optical transparency and suitability for the bacteria staining by nanoparticles (hydrophilicity, no non-specific binding to nanoparticles);

(c) optimized upon changing of a new sample to minimize overlapping staining from previous sample.

The optofluidic chip is capable of detecting a colorimetric and/or fluorescence output emitted from the bacterial cells modified by the reagents in order for the bacterial cells to be quantified relative to a control. In particular, the bacterial cells can be modified with plasmonic and/or fluorescent nanoparticles. The intensity of the coloration and/or fluorescence can be directly proportional to the amount of bacterial cells, and hence used to quantify the amount of bacterial cells in a sample since the sampling volume is known.

In some embodiments, the control is a calibration plot. This involves spiking a sample with a known amount of bacterial cells and passing the sample through the bacterial cell counting device. The plasmonic and/or fluorescent emission can then be directly correlated with the bacterial cell concentration in the sample. Any subsequent samples with unknown amount of bacterial cells can thus be plotted on this calibration plot based on their plasmonic and/or fluorescent emission to determine the bacterial cell concentration in the unknown sample.

Optical sensing of the plasmonic and/or fluorescence emission can be detected either “on-chip” or “off-chip”. In some embodiments, the bacterial cell counting device further comprises means for detecting the colorimetric and/or fluorescence output. The detecting means can be an optical detector for measuring the colorimetric and/or fluorescence output emitted from the bacterial cells modified by the reagents. In this regard, the optical detector is integrated into the bacterial cell counting device.

In particular, the present invention may optically sense plasmonic and/or fluorescence emission via an on-chip approach. In this regard, an LED light source may be installed on the front side of the filter strip, and a photodetector is installed on the back side of the filter strip. Light from the LED is transmitted through the filter strip and captured by the photodetector. Upon bacteria capture and staining with the reagent, the change in the transmitted light may be measured.

In some embodiments, the bacterial cell counting device further comprises a ballast tank. The ballast tank preps the filter strip for receiving the sample. For example, the ballast tank can comprise an aqueous solution for wetting the filter strip and for washing impurities from the filter strip before it receives the sample.

The bacterial cell counting device can further comprises a washing tank. The washing tank comprising an aqueous solution for washing the filter strip after receiving the sample. The washing tank can be configured to have a larger volume than the at least one cartridge. This is in view that, in general, a larger volume is used in a washing step to ensure consistency. The ballast or wetting tank and the washing tank can be combined as a single entity. In this regard, the solution in the ballast tank and the washing tank can be the same; i.e. the solution used to wet the filter strip before receiving the sample and wash the filter strip after receiving the sample can be the same.

The term “aqueous solution” used herein refers to a water based solvent or solvent system, and which comprises of mainly water. Such solvents can be either polar or non-polar, and/or either protic or aprotic. Solvent systems refer to combinations of solvents which resulting in a final single phase. Both ‘solvents’ and ‘solvent systems’ can include, and is not limited to, pentane, cyclopentane, hexane, cyclohexane, benzene, toluene, dioxane, chloroform, diethylether, dichloromethane, tetrahydrofuran, ethyl acetate, acetone, dimethylformamide, acetonitrile, dimethyl sulfoxide, nitromethane, propylene carbonate, formic acid, butanol, isopropanol, propanol, ethanol, methanol, acetic acid, ethylene glycol, diethylene glycol or water. Water based solvent or solvent systems can also include dissolved ions, salts and molecules such as amino acids, proteins, sugars and phospholipids. Such salts may be, but not limited to, sodium chloride, potassium chloride, ammonium acetate, magnesium acetate, magnesium chloride, magnesium sulfate, potassium acetate, potassium chloride, sodium acetate, sodium citrate, zinc chloride, HEPES sodium, calcium chloride, ferric nitrate, sodium bicarbonate, potassium phosphate and sodium phosphate. As such, biological fluids, physiological solutions and culture medium also fall within this definition. In most embodiments, the aqueous solution is water. In some embodiments, the aqueous solution is deionised water. In some embodiments, the aqueous solution is Millipore water.

In some embodiments, the aqueous solution comprises a surfactant. The surfactant may be cationic surfactant, such as cetyltrimethylammonium bromide (CTAB) and cetyltrimethylammonium chloride (CTAC). The surfactant may be a nonionic surfactant such as Tween-20.

In some embodiments, the bacterial cell counting device further comprises a waste collection tank for containing the reagents and sample after elution from the optofluidic chip. The waste collection tank can be sized to contain eluted waste of several samplings. For example, the waste collection tank can of a similar size relative to the washing tank or larger.

In some embodiments, the bacterial cell counting device further comprises means for quantifying the colorimetric and/or fluorescence output. This can be provided by a software with reference to a control.

In some embodiments, the device is characterised by a limit of detection of about 1000 CFU/mL.

In some embodiments, the bacterial cell is selected from E. coli, or S. aureus.

The optofluidic chip is optimized in the following ways: optically, to produce the best signal to noise ratio; fluidically, to allow liquid to flow and cover the maximum area available on the cassette tape; and mechanically, to clamp as tightly as possible on the filter tape without leaking liquid. The optofluidic chip, is particularly designed for direct measurement of the color for bacteria detection on the filter tape.

The bacterial cell counting device further comprises a pumping system for allowing a sequential flow of reagents and sample to the filter strip. The pumping system can comprise a single pump with valves to control the flow of sample and/or reagents, or can comprise multiple pumps each of which controls the flow of sample and reagents.

The bacterial cell counting device comprises a controller or electronic components for controlling a flow of reagents and sample sequentially to the filter strip via the optofluidic chip. This can be performed by the controller controlling a motor or control of pumps, and any necessary valving, for driving or pumping reagents from reservoirs. The controller can also be configured to determine the amount of sample and/or reagents to be injected or flow through the device. This can be performed via the duration of pump (and valve) activation. This improves a user's experience as the lag time between reaction steps can be minimized. The controller can be configured to wind the filter tape by a pre-determined length—e.g. the controller may control a servomotor to advance the tape a predetermined length. This can be performed between periods of pump activation. When the flow is automated to occur sequentially, there is also less chance of user error, thus improving accuracy. The quantifying means and/or the controller can configure the bacterial cell counting device with rules such that a substantial number of steps in detecting and/or quantifying the bacterial cells can be automated. In this regard, and in some embodiments, the bacterial cell counting device is automated. This improves the user's experience and minimizes human error.

For example, the optofluidic chip can be:

(a) incorporated with 3-dimensional microfluidic channels, in situ real-time optical transmission and/or reflection measurements, clamping and unclamping mechanism for filter membrane, chamber pressurization, channel mixing and purging.

(b) optimized to allow smooth processing and fitting to the whole automated system.

(c) integrated with an optical sensor by using on chip digital light metrology.

Accordingly, the present invention also provides a bacterial cell counting system, comprising:

a) the bacterial cell counting device as disclosed herein; and

b) a control system operable to automatically and sequentially flow the reagents and sample to the filter strip via the optofluidic chip.

In some embodiments, the filter tape based automatic bacteria counting system uses metal nanoparticle as “stain”. The entire system can be automated without the need of human action. The measurements can be performed within minutes.

The control system can be a computer implemented system. This can involve a computer programme which regulates the activating of pumps, opening and closing of flow valves and pressure valves. The control system can also be regulated to control the sampling, wash and/or incubation durations. The programme can additionally be configured to enable (such as switch on or off) the detecting means and/or quantifying means in order to detect and/or quantify the amount of bacterial cells present on the filter strip. The control system may also be configured with data relating to the control (calibration plot).

For example, the timings of the bacterial cell detection and/or counting method (discussed below) can be automated through the system.

In some embodiments, the “continuous and programmable operation” is rendered by the filter cassette with filter tapes, and other features. The filter cassette with filter tapes plays the role to capture the bacteria cells from one sample to other samples by introducing new membrane tape surfaces after one test. Other features enable the automated counting by storing the staining reagent, supplying samples and nanoreagents, reading the stained filter, and data recording and controlling all the features.

In some embodiments, the automated bacteria-counting system comprising:

(a) a nano-reagent cartridge;

(b) an automated sampler;

(c) an optofluidic chip;

(d) a filter cassette with filter tapes; and

(e) control and measurement electronics.

The present invention also provides a method counting bacterial cells in a sample using a bacterial cell counting device as disclosed herein. In some embodiments, the method comprises a use of an optofluidic chip, with integrated light source, optical sensor, and fluidics.

In some embodiments, the method comprises:

a) flowing the sample in a liquid form through the filter strip, the filter strip for trapping or retaining bacterial cells on its surface thereof;

b) flowing a first aqueous nanoparticle solution through the filter strip, the first aqueous nanoparticle solution comprising a plasmonic and/or fluorescent nanoparticle and a first surfactant, the nanoparticle functionalised with positively charged molecules and/or affinity probe for binding to said bacterial cells trapped or retained on the surface of the substrate via charge recognition and/or affinity binding; and

c) flowing a second aqueous solution through the filter strip, the second aqueous solution for washing the unbound nanoparticle from the porous substrate; wherein the bacterial cells in the sample is quantifiable by a colorimetric and/or fluorescence output emitted from the nanoparticle bound to the bacterial cells trapped or retained on the filter strip.

In some embodiments, the method comprises:

a) flowing the sample in a liquid form through the filter strip, the filter strip for trapping or retaining bacterial cells on its surface thereof;

b) flowing a first aqueous nanoparticle solution through the filter strip, the first aqueous nanoparticle solution comprising a plasmonic and/or fluorescent nanoparticle and a first ionic surfactant, the nanoparticle functionalised with positively charged molecules and/or affinity probe for binding to said bacterial cells trapped or retained on the surface of the substrate via charge recognition and/or affinity binding; and

c) flowing a second aqueous solution through the filter strip, the second aqueous solution for washing the unbound nanoparticle from the porous substrate;

wherein the bacterial cells in the sample is quantifiable by a colorimetric and/or fluorescence output emitted from the nanoparticle bound to the bacterial cells trapped or retained on the filter strip.

In some embodiments, the method further comprises a step before step a) of purging the filter strip with a wetting solution for removing residual reagents in at least the optofluidic chip. The wetting solution can come from the ballast or wetting tank. The purging step also cleans and preps the filter strip for receiving the sample. The purge can be performed using an aqueous solution. In some embodiments, the purging step is performed for about 1 min to about 5 min, or preferably for less than about 1 min.

The wetting solution may be an aqueous medium, or preferably water.

In some embodiments, the step of flowing the sample is performed for about 1 min to about 5 min, or preferably less than about 3 min. This captures the bacterial cells onto the filter strip.

In some embodiments, the method further comprises a step after step a) of washing filter strip in order to remove non-bacterial cell particulates. The washing solution can come from the washing tank. This further preps the filter strip for receiving the reagents. In some embodiments, the washing step is performed for about 1 min to about 5 min, or preferably less than about 3 min. The washing solution may be an aqueous medium comprising a surfactant. The surfactant may be Tween-20 at a concentration of about 0.5 wt % to about 1 wt %. The washing solution may be water.

In some embodiments, the step of flowing a first aqueous nanoparticle solution is performed for about 1 min to about 5 min, or preferably less than about 3 min. This stains the bacterial cells with the nanoparticle.

In some embodiments, the method further comprises a step after step b) of incubating the first aqueous nanoparticle solution with the bacterial cells trapped or retained on the filter strip. The incubation step can be performed for about 1 min to about 30 min. Preferably, the duration is less than about 15 min, or less than about 10 min.

In some embodiments, the step of flowing a second aqueous solution is performed for about 1 min to about 5 min, or preferably less than about 3 min.

In some embodiments, the method further comprises a step after step c) of detecting and/or quantifying the bacterial cells trapped or retained on the surface of the filter strip.

The method can be regulated by a controller such that the flow of reagents and sample flows sequentially. In some embodiments, the method is automated for sampling and staining on the filter membrane in the cassette.

The present invention also provides a method of counting bacterial cells in a sample, comprising:

a) purging a filter strip with a wetting solution;

b) flowing the sample in a liquid form through the filter strip, the filter strip for trapping or retaining bacterial cells on its surface thereof;

c) flowing a first aqueous nanoparticle solution through the filter strip, the first aqueous nanoparticle solution comprising a plasmonic and/or fluorescent nanoparticle and a first surfactant, the nanoparticle functionalised with positively charged molecules and/or affinity probe for binding to said bacterial cells trapped or retained on the surface of the substrate via charge recognition and/or affinity binding; and

d) flowing a second aqueous solution through the filter strip, the second aqueous solution for washing the unbound nanoparticle from the porous substrate; wherein the bacterial cells in the sample is quantifiable by a colorimetric and/or fluorescence output emitted from the nanoparticle bound to the bacterial cells trapped or retained on the filter strip.

In some embodiment, the method of counting bacterial cells in a sample comprises:

a) purging a filter strip with a wetting solution;

b) flowing the sample in a liquid form through the filter strip, the filter strip for trapping or retaining bacterial cells on its surface thereof;

c) flowing a first aqueous nanoparticle solution through the filter strip, the first aqueous nanoparticle solution comprising a plasmonic and/or fluorescent nanoparticle and a first ionic surfactant, the nanoparticle functionalised with positively charged molecules and/or affinity probe for binding to said bacterial cells trapped or retained on the surface of the substrate via charge recognition and/or affinity binding; and

d) flowing a second aqueous solution through the filter strip, the second aqueous solution for washing the unbound nanoparticle from the porous substrate;

wherein the bacterial cells in the sample is quantifiable by a colorimetric and/or fluorescence output emitted from the nanoparticle bound to the bacterial cells trapped or retained on the filter strip.

In some embodiments, the process for detecting and/or counting bacterial cells comprises:

i) flowing the sample through the filter strip for about 3 min in order to trap or retain bacterial cells on the surface of the filter strip;

ii) flowing a first aqueous solution in order to wash the trapped or retained bacterial cells for about 3 min;

iii) flowing a reagent for about 40 sec in order to stain the bacterial cells;

iv) incubating the stained bacterial cells for about 10 min; and

v) flowing a second aqueous solution for about 3 min in order to wash away the unreacted reagent.

The first and second aqueous solution can be the same.

In some embodiments, the process for detecting and/or counting bacterial cells comprises:

i) purging the filter strip for about 1 min;

ii) flowing the sample through the filter strip for less than about 3 min in order to trap or retain bacterial cells on the surface of the filter strip;

iii) flowing a first aqueous solution for less than about 3 min in order to wash the trapped or retained bacterial cells;

iv) flowing a reagent for about 25 sec in order to stain the bacterial cells;

v) incubating the stained bacterial cells for about 10 min; and

vi) flowing a second aqueous solution for less than about 3 min in order to wash away the unreacted reagent.

In some embodiments, the process for detecting and/or counting bacterial cells comprises:

i) purging the filter strip for about 5 sec;

ii) flowing the sample through the filter strip for about 20 sec in order to trap or retain bacterial cells on the surface of the filter strip;

iii) flowing a first aqueous solution for about 5 sec in order to wash the trapped or retained bacterial cells;

iv) flowing a reagent for about 5 sec in order to stain the bacterial cells;

v) incubating the stained bacterial cells for about 50 sec; and

vi) flowing a second aqueous solution for about 20 sec in order to wash away the unreacted reagent.

In some embodiments, the bacterial cells in a sample is quantifiable within about 1 min to about 30 min. In other embodiments, the bacterial cells is quantifiable within about 1 min to about 25 min, about 1 min to about 20 min, about 1 min to about 15 min, about 1 min to about 10 min, or about 1 min to about 5 min.

The present invention is also directed to a method of counting bacteria by the above automated bacteria-counting system, the method comprising the following steps:

(i) a purging step for purging bacteria cells carried forward from a previous step;

(ii) a capturing step, which includes drawing and filtering a water sample for capturing the bacteria cells in the water sample on a membrane;

(iii) a washing step for removal of sample background (e.g. salt, minerals, or smaller particulates) that may interfere with the binding of the nanoparticle reagent to the bacteria cells

(iv) a staining step, which includes drawing a nanoparticle reagent for staining the bacteria cells on the membrane;

(v) an incubation step to allow the nanoparticles to bind to the bacteria cells;

(vi) a rinsing step for minimizing accumulation of excess nanoparticle reagent not used for staining the bacteria cells;

(vii) a measuring step for measuring the color intensity of the stained bacteria cells and correlating with the bacteria count.

Further, the method is implemented by a software system which allows point and click operation, full programmable recipes (steps, frequency, number of samples), remote controlling, indicator that tells the current operation/sample processed, and API-interfacing.

Overall, the development of the bacterial cell counting device and system can provide the following:

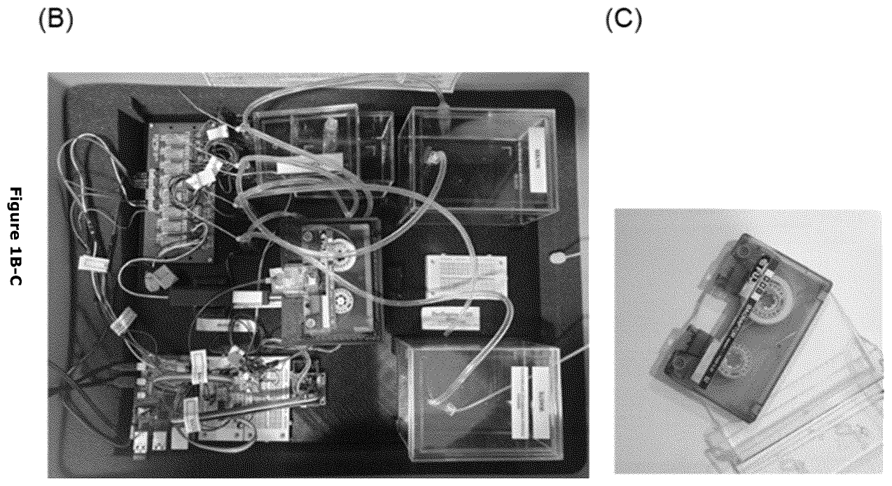

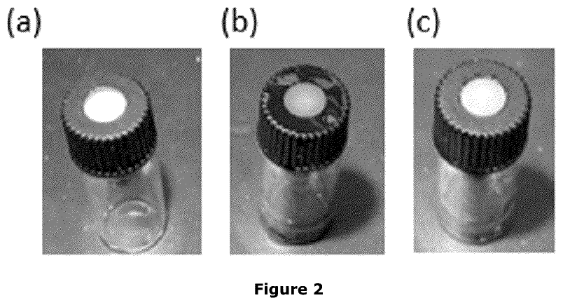

i) A cassette based design which enables a protocol that originally involves multiple consumables (such as solutions, syringes, disposable syringe filters), and multiple manual processes. The cassette has a standardized size for our designed system and is interchangeable with a new cassette. The cassette is loaded with 0.22 μm PVDF membrane, which has been selected and validated for its optical transparency and suitability for the bacteria staining by nanoparticles (hydrophilicity, no non-specific binding to nanoparticles) as shown in FIG. 2. The tape increment upon changing to a new sample is optimized to minimize overlapping staining from previous sample.

ii) A “Optofluidic Chip (OFC)”, which incorporates 3-dimensional microfluidic channels, in situ real-time optical transmission and/or reflection measurements, clamping and unclamping mechanism for filter membrane, chamber pressurization, channel mixing and purging. The design and fabrication of the OFC can be optimized to allow smooth processing and fitting to the whole automated system.

iii) Optical sensor or detector integrated into the OFC by using on chip digital light metrology.

iv) Auto sampling by using multiple mini-sized piezo electric pumps with digital control.

v) A software system allows point and click operation, full programmable recipes (steps, frequency, number of samples), remote controlling, indicator that tells the current operation/sample processed, and API-interfacing.

vi) The bacterial cell counting device can be packaged on a monolithic 3-D printed board and placed within a structural foam case for ease of transport and deployment (smaller than a cabin luggage size).

vii) Testing and optimization of the steps. Particularly, different from the manual process, a “purging” step is included to prevent bacteria cells being carried over from past samples to next sample. A cleaning step is included to minimize accumulation of AuNPs in OFC. As such the whole process (purging, capturing, washing, staining, incubation, rinsing) can increase the accuracy of quantification. The time taken for each step, and the pump flow rate can also be optimized to get the best staining for bacteria detection.

The bacterial cell counting device can be packaged such that convenience is provided to the user. For example,

(a) the bacterial cell counting device can be provided with an auto sampler comprising multiple mini-sized piezo electric pumps with digital control;

(b) the bacterial cell counting device can be packaged on a monolithic 3-D printed board and placed within a structural foam case for ease of transport and deployment;

(c) a cartridge in which the nanoreagent is Au nanoparticles is provided together with the bacterial cell counting device;

(d) the bacterial cell counting device is optimised for use to detect and quantify E coli.

Other bacterial cells can also be detected and quantified. For example, E. coli, S. aureus, and general bacteria from environment can be detected and quantified.

### EXAMPLES

Full demonstration and validation of the bacterial cell counting device and system for continuous measurement of bacteria samples, refereeing the bacteria culture test.

### Demonstration 1: Optofluidic Chip Validation

The optofluidic chip is designed to measure amount of light transmitted through the stained sample. FIG. 3 below is a testing of the detector on marker pen stained PVDF filter membrane. The measured light transmission correlates well with the darkness of the stained spots.

### Demonstration 2. E. coli Counting—“Auto Testing” With Manual Change of Samples

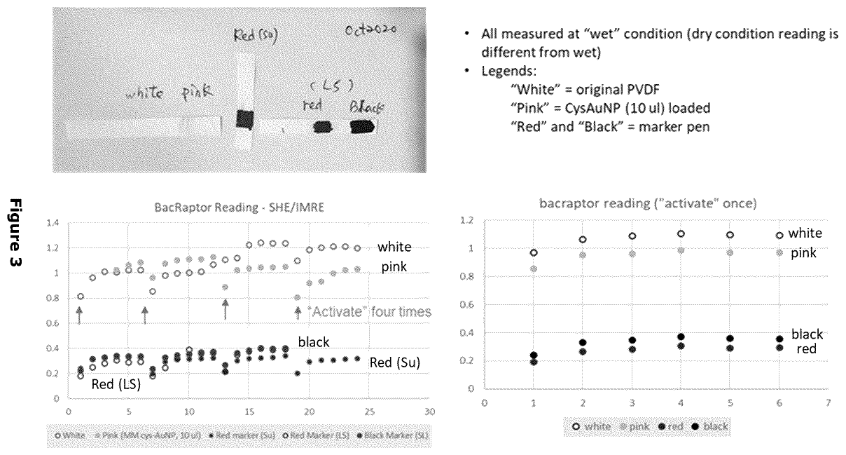

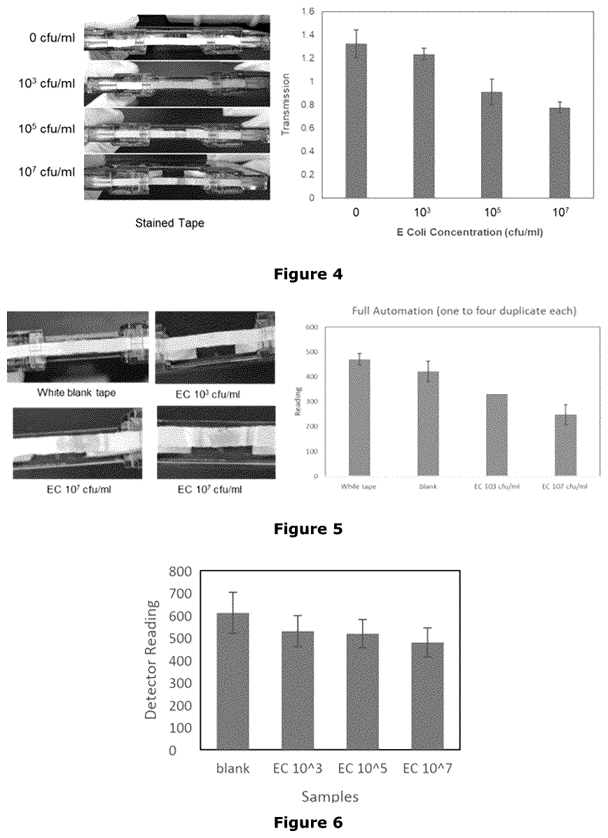

In this demonstration, E. coli samples are added into the sample chamber. The machine is set for single sample measurement using the “Take Measurement” programme. The action steps and their duration are shown in the Table 1. The automation for all of the action steps was set for each sample. At the end of each sample test, the filter tape was moved to the next position by manual instruction through the control software. For each bacteria concentration, the test was repeated twice. FIG. 4 shows the results.

TABLE 1
E Coli counting programme (flow rate 0.33 ml/min)

| | Step | | | | | | |
|---|---|---|---|---|---|---|---|
| | | | | | | | 7 |
| | 1 | 2 | 3 | 4 | 5 | 6 | Measure |
| | Purge | Capture | Wash | Stain | Incubation | Rinse | (pts/sample) |
| Time | 0 | 180 | 180 | 40 | 600 | 180 | 5 |
| (s) | | | | | | | |

### Demonstration 3. Full Automation—Test 1

In this demonstration, the machine is set for continuous multiple sample measurement using the “Automatic Measurement” programme. The action steps and their duration are shown in the table below. For each bacteria concentration, three samples are automatically measured continuously. Bacteria concentration can be detected as low as 1000 CFU/mL.

TABLE 2
Automation programme

| | Step | | | | | | | |
|---|---|---|---|---|---|---|---|---|
| | | | | | | | 7 | 8 |
| | | | | | 5 | | Number | Measure |
| | 1 | 2 | 3 | 4 | In- | 6 | of | (pts/ |
| | Purge | Capture | Wash | Stain | cubation | Rinse | samples | sample) |
| Time | 0 | 180 | 180 | 40 | 600 | 180 | 3 | 5 |
| (s) | | | | | | | | |

### Demonstration 4. Full Automation-Test 2 for 26 Samples

In this demonstration, the machine is set in continuous multiple sample measurement using the “Automatic Measurement” programme for larger number of samples (26 samples). The action steps and their duration are shown in Table 3 below. The bacteria concentration tested was changed without stopping the “Automatic Measurement” programme. The reading results for each bacteria concentration is shown in FIG. 6.

TABLE 3
Automation programme-26 samples

| | Step | | | | | | | |
|---|---|---|---|---|---|---|---|---|
| | | | | | | | 7 | 8 |
| | | | | | 5 | | Number | Measure |
| | 1 | 2 | 3 | 4 | In- | 6 | of | (pts/ |
| | Purge | Capture | Wash | Stain | cubation | Rinse | samples | sample) |
| Time | 60 | 150 | 150 | 24 | 600 | 150 | 26 | 5 |
| (s) | | | | | | | | |

### Demonstration 5. Accelerated Full Automation Test for 50 Samples

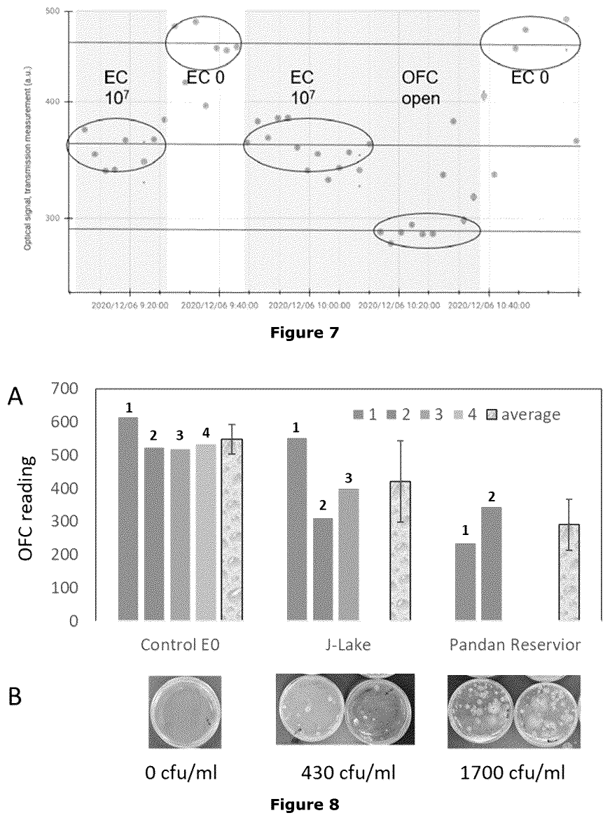

In this demonstration, the machine is tested for full automation of 50 samples in accelerated mode (faster time for each measurement). The action steps and their duration are shown in Table 4 below. The sample is exchanged between 10⁷ CFU/mL of E. coli (EC 107) and water (no bacteria, EC 0). The samples were changed without stopping the “Automation” programme. The reading of switching between 10⁷ CFU/mL of E. coli (EC 107) and EC 0 are shown in FIG. 7. Consistent signal changes was observed responding to the sample switch. The “OFC open” reading (no closure of the optofluidic chip) is also consistent, that can serve as a reference for checking the proper closure of the OFC during the sample testing.

TABLE 4
Automation programme-accelerated, 50 samples

| | Step | | | | | | | |
|---|---|---|---|---|---|---|---|---|
| | | | | | | | | 8 |
| | 1 | 2 | 3 | 4 | 5 | 6 | 7 | Measure |
| | Purge | Capture | Wash | Stain | Incubation | Rinse | Number of samples | (pts/sample) |
| Time | 5 | 20 | 5 | 5 | 50 | 20 | 50 | 4 |
| (s) | | | | | | | | |

### Demonstration 6. Automated Measurement of Lake and Reservoir Water for Total Bacteria Count

In this demonstration, the machine was used to test two real water samples, each from Pandan Reservoir and Journal Lake (sampled in end January 2021). Each water sample was firstly filtered using 5 μm membrane to remove possible particular residues; and then tested multiple times for bacteria count using the machine (the action steps and their duration are shown in Table 5 below). The control test was performed for the real water further filtered through 0.22 μm membrane to get read of bacteria. The same water samples were cultured for 24 hours to get the bacteria count. FIG. 8 shows the results from the bacterial cell counting device and the cultured plate. Both the bacterial cell counting device and culture plate methods show that the Pandan Reservoir water has a higher bacteria count than the Jurong Lake water.

TABLE 5
Process parameters for real water test

| | Step | | | | | | | |
|---|---|---|---|---|---|---|---|---|
| | | | | | | | 7 | |
| | 1 | 2 | 3 | 4 | 5 | 6 | Number | 8 |
| | Purge | Capture | wash | stain | Incubation | Rinse | of samples | Measure (pts/sample) |
| Time(s) | 20 | 120 | 20 | 40 | 60 | 40 | 3 | 10 |

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A bacterial cell counting device, comprising:
a) at least one cartridge for containing reagents;
b) an inlet for introducing a sample containing bacterial cells into the device;
c) an optofluidic chip separately in fluid communication with the cartridge and the inlet;
d) a filter strip passing through the optofluidic chip and in fluid communication with the cartridge and the inlet, the filter strip for trapping or retaining bacterial cells on its surface such that the bacterial cells can interact with the reagents as they flow through the filter strip; and
e) a controller for controlling a sequential flow of reagents and sample to the filter strip via the optofluidic chip;
wherein the optofluidic chip is capable of detecting a colorimetric and/or fluorescence output emitted from the bacterial cells modified by the reagents in order for the bacterial cells to be quantified relative to a control.

2. The bacterial cell counting device according to claim 1, wherein the cartridge is replaceable.

3. The bacterial cell counting device according to claim 1, wherein the at least one cartridge comprises an aqueous nanoparticle solution, wherein the aqueous nanoparticle solution comprising a plasmonic and/or fluorescent nanoparticle and a surfactant, the nanoparticle functionalised with positively charged molecules and/or affinity probes for binding to said bacterial cells trapped or retained on the surface of the filter strip via charge recognition and/or affinity binding.

4. (canceled)

5. The bacterial cell counting device according to a claim 1, further comprising a second cartridge,
wherein the at least one cartridge comprises an aqueous nanoparticle solution comprising a plasmonic and/or fluorescent nanoparticle functionalised with positively charged molecules and a surfactant; and
wherein the second cartridge comprises an aqueous nanoparticle solution comprising a plasmonic and/or fluorescent nanoparticle functionalised with affinity probes and a second surfactant.

6. The bacterial cell counting device according to claim 1, further comprising a washing tank, the washing tank comprising an aqueous solution for washing the filter strip with bacterial cells trapped or retained on its surface.

7. The bacterial cell counting device according to claim 1, wherein the optofluidic chip is a microfluidic chip.

8. The bacterial cell counting device according claim 1, wherein the optofluidic chip further comprises at least one of the following:
i) clamping and unclamping means for securing and releasing the filter strip;
ii) means for chamber pressurization;
iii) means for channel mixing; and
iv) purging means.

9. The bacterial cell counting device according to claim 1, wherein the filter strip is provided as a filter tape in a filter cassette, wherein the filter tape is windable to an adjacent unused region at a predetermined distance from a used region.

10. (canceled)

11. The bacterial cell counting device according to claim 9, wherein when the filter tape comprises at least two used regions, an earlier used region is recallable for measurement by winding the filter tape to that earlier used region.

12. The bacterial cell counting device according to claim 1, wherein the filter strip is a PVDF membrane having a pore size of about 0.22 μm.

13. The bacterial cell counting device according to claim 1, further comprising a waste collection tank for containing the reagents and sample after elution from the optofluidic chip.

14. The bacterial cell counting device according to claim 1, further comprising an optical detector for measuring the colorimetric and/or fluorescence output emitted from the bacterial cells modified by the reagents.

15. The bacterial cell counting device according to claim 1, wherein the control is a calibration plot.

16. The bacterial cell counting device according to claim 1, wherein the bacterial cell counting device is characterised by a limit of detection of about 1000 CFU/mL.

17. A bacterial cell counting system, comprising:
a) the bacterial cell counting device according to claim 1; and
b) a control system operable to automatically and sequentially flow the reagents and sample to the filter strip via the optofluidic chip.

18. A method of counting bacterial cells in a sample using a bacterial cell counting device according to claim 1, comprising:
a) flowing the sample in a liquid form through the filter strip, the filter strip for trapping or retaining bacterial cells on its surface thereof;
b) flowing a first aqueous nanoparticle solution through the filter strip, the first aqueous nanoparticle solution comprising a plasmonic and/or fluorescent nanoparticle and a first ionic surfactant, the nanoparticle functionalised with positively charged molecules and/or affinity probe for binding to said bacterial cells trapped or retained on the surface of the substrate via charge recognition and/or affinity binding; and
c) flowing a second aqueous solution through the filter strip, the second aqueous solution for washing the unbound nanoparticle from the porous substrate;
wherein the bacterial cells in the sample is quantifiable by a colorimetric and/or fluorescence output emitted from the nanoparticle bound to the bacterial cells trapped or retained on the filter strip.

19. The method according to claim 18, further comprising a step before step a) of purging the filter strip with a wetting solution from a ballast tank for removing residual reagents in at least the optofluidic chip.

20. The method according to claim 18, further comprising a step after step a) of washing filter strip in order to remove non-bacterial cell particulates.

21. The method according to claim 18, further comprising a step after step b) of incubating the first aqueous nanoparticle solution with the bacterial cells trapped or retained on the filter strip.

22. The method according to claim 18, wherein the bacterial cells in the sample are quantifiable within about 1 min to about 30 min.
